(19) United States
(12) Patent Application Publication
(10) Pub. No.: US 2025/0169727 A1
(43) Pub. Date: May 29, 2025

(54) METHOD AND AN ANALYTE SENSOR SYSTEM FOR DETECTING AT LEAST ONE ANALYTE

(71) Applicant: Roche Diabetes Care, Inc., Indianapolis, IN (US)

(72) Inventor: Kirill Sliozberg, Mannheim (DE)

(21) Appl. No.: 19/036,983

(22) Filed: Jan. 24, 2025

Related U.S. Application Data
(63) Continuation of application No. PCT/EP2023/070407, filed on Jul. 24, 2023.

(30) Foreign Application Priority Data
Jul. 26, 2022 (EP) 22 186 884.7

(51) Int. Cl. A61B 5/1468; A61B 5/00; A61B 5/145; G16H 40/63

(52) U.S. Cl. CPC A61B 5/1468; A61B 5/14532; A61B 5/14546; A61B 5/7264; G16H 40/63; A61B 2560/0468; A61B 2562/0215

(57) ABSTRACT

Disclosed is a method of continuously in vivo detecting an analyte in a bodily fluid over time, an analyte sensor system for in vivo continuously detecting an analyte in a bodily fluid over a measurement time span, and a computer program and a computer-readable storage medium. The method uses an analyte sensor with a working electrode that performs an electrochemical detection reaction with the analyte, and a further electrode having a redox material composition with silver and silver chloride. The method includes the following steps: monitoring a standard sensor signal derived by using the analyte sensor in a standard operation mode, comparing the standard sensor signal with a threshold to thereby determine if a change of the analyte sensor from the standard operation mode into an economy operation mode is required.

### RELATED APPLICATIONS

This application is a continuation of International Application Serial No. PCT/EP2023/070407, filed Jul. 24, 2023, which claims priority to European Patent Application Serial No. 22 186 884.7, filed Jul. 26, 2022, both of which are hereby incorporated herein by reference.

### BACKGROUND

This disclosure relates to a method of continuously in vivo detecting at least one analyte in a bodily fluid over a time span. This disclosure further relates to an analyte sensor system for in vivo continuously detecting at least one analyte in a bodily fluid over a measurement time span. This disclosure further relates to a computer program and a computer-readable storage medium. The method and device of this disclosure, as an example, may be used for diagnostic purposes, e.g., in clinical or laboratory analytics or for home monitoring purposes. The method and device of this disclosure specifically may be used for detecting at least one analyte in a bodily fluid or other liquids.

A wide variety of analyte sensors for detecting at least one analyte in at least one fluid sample, particularly in a bodily fluid, have been described. Analyte sensors configured for reliably detecting chemical and/or biological species in a qualitative and/or quantitative manner can be used for various purposes such as, but not limited to, diagnostic purposes, monitoring of environmental contamination, food safety evaluation, quality control or manufacturing processes.

Analyte sensors for detecting at least one analyte may be fully or partially implanted or inserted in a body tissue. The at least one analyte may be detected in an electrochemical detection reaction occurring between electrodes. For driving such a reaction, generally at least one of the electrodes may comprise a redox material composition comprising a silver compound, specifically silver or silver chloride. An example, for such a redox species may be molecular oxygen dissolved in an interstitial fluid, which may get reduced to an ionic species. Using such a type of counter and/or auxiliary electrode may be typical for a Libre system and may demand the use of an additional reference electrode.

When implanted into a body tissue, however, silver chloride may cause an immune response of the human body, which may lead to a temporary or permanent malfunction of the at least one analyte sensor. It is therefore generally desirable to keep the amount of silver chloride in the electrodes, particularly the amount of silver chloride that is in touch with body tissue, as low as possible, in order to minimize these negative effects.

However, the amount of silver chloride typically cannot be reduced arbitrarily. Rather, other boundary conditions must be taken into account. In that sense, it has to be considered that during the operation of the analyte sensor, the silver chloride is typically consumed in a second electrochemical half-reaction that may take place at the counter or at the combined reference-counter electrode. The consumed amount of silver chloride typically depends directly on the current between the electrodes and the current generally depends on the quantity of the detected analyte. Thus, the consumed amount of silver chloride generally depends directly on the quantity of the detected analyte. As a result, generally, the higher the amount of the analyte to be detected, the higher the consumption of silver chloride. The consumed amount is, thus, generally particularly high during a hyperglycemic period, in which a user or patient has a dangerously high blood glucose level. The minimum amount of silver chloride comprised by the electrode typically has to be calculated for a worst-case-scenario, in which a high amount of analytes in multiple hyperglycemic period may have to be assumed. Only then the analyte sensor can be used by any patient for a sufficiently long time span. As a result, the electrode has to comprise a minimum amount of silver chloride.

In the state of the art, different approaches for designing and operating analyte sensors with a reduced amount of silver chloride in a redox material composition are known.

Richard D. Beach, Robert W. Conlan, Markham C. Godwin, and Francis Moussy, Towards a Miniature Implantable In Vivo Telemetry Monitoring System Dynamically Configurable as a Potentiostat or Galvanostat for Two-and Three-Electrode Biosensors, IEEE TRANSACTIONS ON INSTRUMENTATION AND MEASUREMENT, Volume: 54, Issue: 1, Feb. 2005, Page(s): 61-72 describes a miniature implantable and dynamically configurable potentiostat and galvanostat for two-and three-electrode biosensors with a telemetry electronics package was developed to provide remote monitoring of implantable amperometric and voltametric biosensors such as for glucose. Included are circuitry for sensor biasing, a transimpedance amplifier to produce the sensor proportional output signal, and a transceiver (transmitter and receiver) which can both receive setup parameter values and transmit the biosensor concentration data to a corresponding remote transceiver and computer for monitoring. Remotely configurable features included in the in vivo implanted unit include: sensor excitation changes; filter frequency cutoff values, amplifier gain; and transmission intervals utilizing 303.825-MHz UHF RF telemetry for end-to-end remote data monitoring. The developed mP/Gstat printed circuit board measures about 51 22 1 mm thick, and is suitable for bench top or encapsulated use.

U.S. Publication No. 2021/0030338 A1 discloses a biosensor for measuring a physiological signal representative of a physiological parameter associated with an analyte. The biosensor includes a working electrode and a counter electrode. The counter electrode including a silver and a silver halide having an initial amount, wherein the initial amount is determined by the following steps of defining a required consumption range of the silver halide during at least one of the measurement periods performed by the biosensor; and determining the initial amount based on a sum of an upper limit of the required consumption range and a buffer amount, so that a required replenishment amount range of the silver halide during the replenishment period is controlled to be sufficient to maintain an amount of the silver halide within a safe storage range.

Despite the advantages achieved by known methods and devices as discussed above, several technical challenges remain. Specifically, the consumption of silver chloride in many cases still remains a limiting factor for the duration of use of in vivo sensors.

### SUMMARY

This disclosure teaches methods and devices which at least partially address the above-identified technical challenges. Specifically, a method and an analyte sensor system for in vivo continuously detecting at least one analyte in a bodily fluid over a measurement time span are disclosed which address the problem of the limitation of sensor lifetime by the consumption of the redox material composition. It is generally desirable to further reduce the amount of the redox material composition without sacrificing the operation lifetime of the sensor system.

As used in the following, the terms “have,” “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B,” “A comprises B” and “A includes B” may both refer to a situation in which, besides B, no other element is present in A (i.e., a situation in which A solely and exclusively consists of B) and to a situation in which, besides B, one or more further elements are present in entity A, such as element C, elements C and D or even further elements.

Further, it shall be noted that the terms “at least one,” “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once. It shall also be understood for purposes of this disclosure and appended claims that, regardless of whether the phrases “one or more” or “at least one” precede an element or feature appearing in this disclosure or claims, such element or feature shall not receive a singular interpretation unless it is made explicit herein. By way of non-limiting example, the terms “analyte,” “sensor,” and “sensor signal,” to name just a few, should be interpreted wherever they appear in this disclosure and claims to mean “at least one” or “one or more” regardless of whether they are introduced with the expressions “at least one” or “one or more.” All other terms used herein should be similarly interpreted unless it is made explicit that a singular interpretation is intended.

Further, as used in the following, the terms “preferably,” “more preferably,” “particularly,” “more particularly,” “specifically,” “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

In a first aspect of this disclosure, a method of continuously in vivo detecting at least one analyte in a bodily fluid over a time span is disclosed. The method is using at least one analyte sensor comprising at least one working electrode, configured for performing at least one electrochemical detection reaction with the analyte, and at least one further electrode, the further electrode comprising at least one redox material composition, the redox material composition comprising silver and silver chloride.

The methods comprises the following steps, which may be performed in the given order. A different order, however, is also feasible. Further, two or more of the method steps may be performed simultaneously or in a fashion overlapping in time. Further, the method steps may be performed once or repeatedly. Thus, one or more or even all of the method steps may be performed once or repeatedly. The method may comprise additional method steps which are not listed herein.

The method comprises the following steps:
i. monitoring at least one standard sensor signal derived by using the analyte sensor in a standard operation mode, wherein, in the standard operation mode, potentiostatic measurements are performed for detecting the at least one analyte with the analyte sensor, wherein a potential of the working electrode with respect to the further electrode is set to at least one predetermined standard operating potential and wherein an current through the working electrode and the further electrode is determined, particularly wherein the predetermined standard operating potential is kept constant during the determination of the current; and
ii. comparing the standard sensor signal with at least one threshold, specifically for determining if the standard sensor signal exceeds the threshold, thereby determining if a change of an operation mode of the analyte sensor from the standard operation mode into an economy operation mode is required.

The term “analyte” as used herein is a broad term and is to be given its ordinary and customary meaning to a person of ordinary skill in the art and is not to be limited to a special or customized meaning. The term specifically may refer, without limitation, to an arbitrary chemical or biological substance or species, such as an ion, an atom, a molecule or a chemical compound. The analyte specifically may be an analyte, which may be present in a bodily fluid or a body tissue. The term analyte specifically may encompass atoms, ions, molecules and macromolecules, in particular biological macromolecules such as nucleic acids, peptides and proteins, lipids, sugars, such as glucose, and metabolites. Further examples of potential analytes to be detected will be given in further detail below.

The term “analyte sensor” as used herein is a broad term and is to be given its ordinary and customary meaning to a person of ordinary skill in the art and is not to be limited to a special or customized meaning. The term specifically may refer, without limitation, to an arbitrary device being configured for detecting the at least one analyte by acquiring at least one sensor signal, specifically a standard sensor signal or an economy sensor signal. Alternatively or in addition the sensor signal may be read-out from an electronic unit. As particularly preferred, the analyte sensor may be configured as a fully implantable analyte or a partially implantable analyte sensor which may, particularly, be adapted for performing the detection of the analyte in a bodily fluid of a user in a subcutaneous tissue.

The term “at least partially implantable analyte sensor” as used herein is a broad term and is to be given its ordinary and customary meaning to a person of ordinary skill in the art and is not to be limited to a special or customized meaning. The term specifically may refer, without limitation, to an analyte sensor designated to be fully or partially introduced into the body tissue of a user in a fashion that a first portion of the implantable analyte sensor may be received by the body tissue while a further portion may or may not be received by the body tissue. For this purpose, the analyte sensor may comprise an insertable portion. The term “insertable portion” as used herein is a broad term and is to be given its ordinary and customary meaning to a person of ordinary skill in the art and is not to be limited to a special or customized meaning. The term specifically may refer, without limitation, to a part or component of the analyte sensor, which is configured for being insertable into an arbitrary body tissue. Other parts or components of the analyte sensor, in particular contact pads, may remain outside of the body tissue. The analyte sensor, specifically, may be a transcutaneously insertable analyte sensor, which may fully or partially be inserted into the body tissue through the skin. During use, the analyte sensor may fully be located in the body tissue beneath the skin, or may partially protrude from the body tissue through the skin. Specifically, a portion of the analyte sensor and/or one or more cables may protrude from the body tissue through the skin, e.g., for electrical contacting purposes.

The terms “user” and “patient” as used herein is a broad term and is to be given its ordinary and customary meaning to a person of ordinary skill in the art and is not to be limited to a special or customized meaning. The term specifically may refer, without limitation, to a human being or an animal, independent from the fact that the human being or animal, respectively, may be in a healthy condition or may suffer from one or more diseases. As an example, the user or the patient may be a human being or an animal suffering from diabetes. However, additionally or alternatively, this disclosure may be applied to other types of users, patients or diseases.

The term “in vivo” as used herein is a broad term and is to be given its ordinary and customary meaning to a person of ordinary skill in the art and is not to be limited to a special or customized meaning. The term specifically may refer, without limitation, to the at least one analyte sensor which is configured for being at least partially implanted into the body tissue of the user. The analyte sensor, specifically, may be a subcutaneous or a transcutaneous analyte sensor. The analyte sensor may be configured for continuous monitoring of the analyte.

As further used herein, the term “bodily fluid,” generally, refers to a fluid, in particular a liquid, which is typically present in a body or a body tissue of the user or the patient and/or may be produced by the body of the user or the patient. Preferably, the bodily fluid may be selected from the group consisting of blood and interstitial fluid. However, additionally or alternatively, one or more other types of bodily fluids may be used, such as saliva, tear fluid, urine or other bodily fluids. During the detection of the at least one analyte, the bodily fluid may be present within the body or body tissue. Thus, the analyte sensor may, specifically, be configured for detecting the at least one analyte within the body tissue. Further examples of potential bodily fluids will be given in further detail below.

In particular, the analyte sensor may be an electrochemical analyte sensor. The term “electrochemical sensor” as used herein is a broad term and is to be given its ordinary and customary meaning to a person of ordinary skill in the art and is not to be limited to a special or customized meaning. The term specifically may refer, without limitation, to an analyte sensor which is adapted for a detection of an electrochemically detectable property of the analyte, such as an electrochemical detection reaction. Thus, for example, the electrochemical detection reaction may be detected by applying and/or comparing one or more electrode potentials and/or by applying one or more electrode currents and detecting one or more electrode potentials or voltages. The electrochemical detection reaction may imply one or more redox reactions taking place at one or more electrodes of the analyte sensor by electrical means. Specifically, the electrochemical analyte sensor may be adapted to generate and/or influence the at least one sensor signal, specifically a standard sensor signal, that may particularly be generated by the analyte sensor, or an economy sensor signal, that may particularly be generated by an electronic unit of the analyte sensor system that is influenced by the analyte sensor. The sensor signal may, directly or indirectly, indicate a presence and/or an extent of the electrochemical detection reaction, such as at least one current signal, particularly in a standard operation mode, and/or at least one voltage signal, particularly in an economy operation mode. Alternatively or in addition, the sensor signal may be read-out from the electronic unit. The measurement may be a qualitative and/or a quantitative measurement. Still, other embodiments are feasible.

The term “working electrode” as used herein is a broad term and is to be given its ordinary and customary meaning to a person of ordinary skill in the art and is not to be limited to a special or customized meaning. The term specifically may refer, without limitation, to an electrode of the analyte sensor which is configured for measuring a signal, such as a volt-age, a current, a charge or an electrical/electrochemical potential, dependent on the degree of an electrochemical detection reaction taking place at the working electrode, for the purpose of detecting the at least one analyte. The working electrode may comprise at least one specific chemical component for enhancing the electrochemical process for a specific analyte. Alternatively or additionally, the working electrode may comprise at least one enzyme for catalyzing at least one reaction, particularly at least one oxidation reaction and/or at least one reduction reaction of the analyte to be detected. Examples of enzymes suited for glucose monitoring are given in J. Hones et al.: “The Technology behind Glucose Meters: Test Strips”, Diabetes Technology & Therapeutics, Volume 10, Supplement 1, 2008, S-10-S-26. Other options, e.g., other options depending of the target analyte to be detected, however, are feasible and generally known to the skilled person in the field of electrochemical analyte detection. As an example, the working electrode may comprise at least one enzyme for catalyzing at least one reaction, wherein the working electrode may oxidize and/or reduce at least one product of the reaction. Such a product may be a further chemical component or at least one electron. The at least one analyte to be detected may further be specified by using at least one specific coating, particularly a membrane which permits permeation specifically for the at least one analyte to be detected.

The term “further electrode” as used herein is a broad term and is to be given its ordinary and customary meaning to a person of ordinary skill in the art and is not to be limited to a special or customized meaning. The term specifically may refer, without limitation, to an electrode that may comprise at least one redox material composition, specifically silver and/or silver chloride. The further electrode may fully or partially be covered with at least one redox material composition. As an example, the further electrode may comprise at least one conductive electrode pad, such as an electrode pad fully or partially made of at least one metal such as gold or platinum, wherein the at least one electrode pad may fully or partially be covered with at least one layer of the redox material composition being part of the further electrode.

The at least one further electrode specifically may comprise at least one counter electrode and/or at least one reference electrode. The term “counter electrode” as used herein is a broad term and is to be given its ordinary and customary meaning to a person of ordinary skill in the art and is not to be limited to a special or customized meaning. The term specifically may refer, without limitation, to an electrode adapted for performing at least one electrochemical counter reaction and/or configured for balancing a current flow due to the detection reaction at the working electrode. The term “reference electrode” as used herein is a broad term and is to be given its ordinary and customary meaning to a person of ordinary skill in the art and is not to be limited to a special or customized meaning. The term specifically may refer, without limitation, to an electrode of the analyte sensor which is configured to provide an electrochemical reference potential which, at least widely, is independent of the presence or absence or concentration of the analyte. The reference electrode may be configured for being a reference for measuring and/or controlling a potential of the working electrode.

Thus, the analyte sensor may be implanted partially or fully into the body tissue, e.g., transcutaneously. Specifically, the at least one working electrode and the at least one further electrode may be located within the body tissue, thereby preferably being in contact with at least one bodily fluid of the user. The term “implanting,” or any grammatical variation thereof, as used herein is a broad term and is to be given its ordinary and customary meaning to a person of ordinary skill in the art and is not to be limited to a special or customized meaning. The term specifically may refer, without limitation, to the process of fully or at least partially inserting an artificial material or an object, into the human body to remain there at least for a longer period of time, specifically at least the time span. The implanting, which may comprise the process of inserting, may comprise, as an example, a transcutaneous insertion, e.g., by using a cannula. Specifically, the implanting may comprise or may be a transcutaneous insertion through the skin of the user, into a body tissue, such as into the interstitium of the user. The process of implantation, thereby, may imply a minor incision into the skin, only, without the necessity of implantation into a blood vessel of the user.

The process of implantation, thus, may be performed without the necessity of a substantial physical intervention on the body requiring professional medical expertise to be carried out and entailing a substantial health risk even when carried out with the required professional care and expertise. As an example, the implantation may include a minor incision into the skin, only, e.g., an incision having a lateral extension of less than 5 mm, e.g., of less than 3 mm. Further, the implantation may include a depth of insertion of the analyte sensor of no more than 30 mm, e.g., of no more than 20 mm, into the body tissue.

Additionally or alternatively, the method may not comprise the step of implanting or inserting the analyte sensor into the body tissue, at all. Thus, as an example, the method may merely be a method of operating the analyte sensor. The method, thus, may not provide any functional interaction with the effects produced by the analyte sensor on the body.

The term “continuously detecting” as used herein is a broad term and is to be given its ordinary and customary meaning to a person of ordinary skill in the art and is not to be limited to a special or customized meaning. The term specifically may refer, without limitation, to a process of detecting a time-series of measurement values of at least one measurement variable. The continuous detecting, specifically, may comprise acquiring and/or recording a series of measurement values at different points in time, e.g., after constant time intervals, with a constant measurement frequency or after irregular time intervals. The continuous detecting may comprise constant, permanent and/or frequently measuring. Thereby, a qualitatively and/or quantitatively evaluation of the at least one analyte possible may be made possible. The measurement may be quantitative, so that a concentration value of the analyte may evaluated over a period of time, also referred to as a time span of the measurement. The measurement may be performed by using the analyte sensor. For such a purpose a sensor signal may be generated, particularly by the analyte sensor and/or the electronic unit. Alternatively or in addition, the sensor signal may be read-out from an electronic unit. The term “over a time span” as used herein is a broad term and is to be given its ordinary and customary meaning to a person of ordinary skill in the art and is not to be limited to a special or customized meaning. The term specifically may refer, without limitation, to detecting the at least one analyte for a period of time, e.g., for a plurality of hours or even a plurality of days or weeks, e.g., for 7 days to 4 weeks.

Further according to the first aspect, the method comprises a step i. According to step i., as outlined above, the method comprises monitoring at least one standard sensor signal derived by using the analyte sensor in a standard operation mode. In the standard operation mode, potentiostatic measurements are performed for detecting the at least one analyte with the analyte sensor, wherein a potential of the working electrode with respect to the further electrode is set to at least one predetermined standard operating potential and wherein an current through the working electrode and the further electrode is determined, particularly wherein the predetermined standard operating potential is kept constant during the determination of the current. The current may be an electric current. The potential may be an electric potential.

The term “monitoring” as used herein is a broad term and is to be given its ordinary and customary meaning to a person of ordinary skill in the art and is not to be limited to a special or customized meaning. The term specifically may refer, without limitation, to a process of continuously acquiring data and deriving desired information therefrom without user interaction. For this purpose, at least one standard sensor signal and/or at least one economy sensor signal, specifically a plurality of sensor signals, may be generated and evaluated, wherefrom the desired information is determined, specifically for determining a required change of the operation mode of the at least one analyte sensor, particularly a change from the standard operation mode to the economy operation mode, or vice versa.

The term “standard operation mode” as used herein is a broad term and is to be given its ordinary and customary meaning to a person of ordinary skill in the art and is not to be limited to a special or customized meaning. The term specifically may refer, without limitation, to a specific operation mode of the at least one analyte sensor. The standard operation mode may be the mode of operation, which is used as a default, e.g., unless at least one exceptional condition is met, as will be outlined below. In the standard mode the at least one analyte may be detected continuously. Therefore, standard sensor signal may be generated, Additionally, a qualitative and/or quantitative evaluation of the at least one analyte may be performed. In the standard operation mode potentiostatic measurements may be performed.

The term “potentiostatic measurement” as used herein is a broad term and is to be given its ordinary and customary meaning to a person of ordinary skill in the art and is not to be limited to a special or customized meaning. The term specifically may refer, without limitation, to a measurement wherein the standard operating potential between the working electrode and the further electrode is predefined, particularly during the detection of the at least one analyte from a plurality of sensor signals, specifically the standard sensor signal. The standard operating potential may be constant at a predefined value, typically of 30 mV to 60 mV or of 50 mV, particularly for an Os-complex modified polymer based mediator. Thereby, the analyte sensor may be operated in a diffusion-controlled current mode, in which the sensor signal, specifically the standard sensor signal, may depend only on the analyte concentration. The standard operating potential may be generated by the electronic unit.

The term “standard sensor signal” as used herein is a broad term and is to be given its ordinary and customary meaning to a person of ordinary skill in the art and is not to be limited to a special or customized meaning. The term specifically may refer, without limitation, to a sensor signal, e.g., a raw signal and/or a pre-processed or processed sensor signal, generated by the analyte sensor when being operated in the standard operation mode. Specifically, the standard sensor signal may be a raw sensor signal or a pre-processed or processed sensor signal being derived by detecting the current flowing between the working electrode and the further electrode, specifically in the standard operation mode. The current may depend on the quantity of the at least one analyte, particularly the concentration of the at least one analyte in the bodily fluid. Additionally or alternatively, the standard sensor signal may be derived from the current, particularly by considering a calibration value and/or calibration function. Thereby, the standard sensor signal may be the glucose level, specifically the blood glucose level.

The term “determining” as used herein is a broad term and is to be given its ordinary and customary meaning to a person of ordinary skill in the art and is not to be limited to a special or customized meaning. The term specifically may refer, without limitation, to a process of measuring and/or generating at least one representative result, in particular, by evaluating the at least one sensor signal as acquired by the analyte sensor and/or the electronic unit, particularly for deriving information about the at least one sensor signal correlated to the at least one analyte for detecting the at least one analyte qualitatively and/or quantitatively.

Further according to the first aspect, the method comprises a step ii., as also outlined above. According to the step ii., the method comprises comparing the standard sensor signal with at least one threshold, specifically for determining if the standard sensor signal exceeds the threshold, thereby determining if a change of an operation mode of the analyte sensor from the standard operation mode into an economy operation mode is required.

The term “comparing” as used herein is a broad term and is to be given its ordinary and customary meaning to a person of ordinary skill in the art and is not to be limited to a special or customized meaning. The term specifically may refer, without limitation, to finding a relationship between the respective sensor signal and the threshold. Thus, when quantifiable items A and B are compared, the result of the comparison may comprise at least one of the following: A is bigger than B; A is equal to B; A is smaller than B. The term “threshold” as used herein is a broad term and is to be given its ordinary and customary meaning to a person of ordinary skill in the art and is not to be limited to a special or customized meaning. The term specifically may refer, without limitation, to at least one predetermined or determinable value of comparison, with which at least one other item or value is compared. As an example, the threshold, in case of electrical signals, may comprise at least one threshold voltage and/or at least one threshold current with which at least one voltage signal or at least one current signal may be compared. In the present case, the threshold may comprise at least one threshold current in the standard operation mode. The threshold current may be calculated by considering at least one of: a sensor sensitivity; or a threshold glucose level, specifically a threshold blood glucose level. For example, for an analyte sensor that may have a sensor sensitivity of 0.05 nA/mg/dl and a threshold glucose level, specifically a threshold blood glucose level, that may be considered to be 250 mg/dl, the threshold current may amount to 12.5 nA. Alternatively or in addition, the threshold current may correspond to a threshold glucose level, specifically a threshold blood glucose level. The threshold glucose level, specifically the threshold blood glucose level, may typically be 200 mg/dl or 300 mg/dl. Thus, the threshold may be determined empirically, e.g., in a laboratory environment, by determining the current which occurs at a predetermined threshold glucose level, specifically a predetermined threshold blood glucose level, e.g., a threshold blood glucose level chosen in the range of 200 mg/dl to 300 mg/dl. A glucose level, specifically a blood glucose level, above the threshold glucose level, specifically the threshold blood glucose level, may be determined to be in the hyperglycemic range.

The term “determining if the economy sensor signal exceeds the economy threshold” as used herein is a broad term and is to be given its ordinary and customary meaning to a person of ordinary skill in the art and is not to be limited to a special or customized meaning. The term specifically may refer, without limitation, to determining if the standard sensor signal, specifically when being the current, exceeds the threshold current, specifically in the standard operation mode. Alternatively or additionally, determining if the standard sensor signal, specifically when being the glucose level, specifically the blood glucose level, exceeds the threshold glucose level, specifically the threshold blood glucose level, particularly in the standard operation mode.

The term “change of an operation mode” as used herein is a broad term and is to be given its ordinary and customary meaning to a person of ordinary skill in the art and is not to be limited to a special or customized meaning. The term specifically may refer, without limitation, to a change or switch of the functionality of the analyte sensor, particularly so that the amount of consumption of the redox material composition, specifically the silver chloride, is changed or switched. The operation modes of the analyte sensor may be at least a standard operation mode and an economy operation mode. There may be further operation modes. The choice and/or the change of operation mode, specifically, may be performed automatically, e.g., computer-implemented, such as by a processor of the analyte sensor system.

The term “economy mode” as used herein is a broad term and is to be given its ordinary and customary meaning to a person of ordinary skill in the art and is not to be limited to a special or customized meaning. The term specifically may refer, without limitation, to a specific operation mode of the at least one analyte sensor. The term specifically may refer, without limitation, to an exceptional operation mode of the at least one analyte sensor which is chosen or initiated when at least one specific condition is fulfilled, e.g., at least one condition selected from the group consisting of: the standard sensor signal being above the threshold; the standard sensor signal being below the threshold; the standard sensor signal being above or being equal to the threshold; the standard sensor signal being below or being equal to the threshold. In the economy mode the at least one analyte may be further detected. Therefore, a qualitative and/or quantitative detection and/or evaluation of the at least one analyte may be performed in the economy mode. Additionally or alternatively, galvanostatic measurements may be performed in the economy mode. This will be described in more detail below.

The sensitivity of the analyte sensor for detecting the at least one analyte, particularly the concentration value of the analyte in the bodily fluid, may be lower in the economy operation mode than in the standard operation mode. The term “sensitivity” as used herein is a broad term and is to be given its ordinary and customary meaning to a person of ordinary skill in the art and is not to be limited to a special or customized meaning. The term specifically may refer, without limitation, to the amount of change in the sensor signal of the analyte sensor per amount of change in the concentration value of the analyte. The current flowing through the working electrode and the further electrode in the standard operation mode may depend on analyte concentration, wherein the sensitivity may typically be given in nA/mg/dl. The analyte concentration in the economy mode may be calculated by considering the operating potential of the working electrode with respect to the further electrode, wherein the sensitivity typically may be given in mV/mg/dl.

The standard sensor signal may comprise information about the at least one analyte, specifically a concentration value, particularly wherein the standard sensor signal may be, specifically exclusively, analyzed for detecting the at least one analyte in the standard operation mode. The term “comprise information about the at least one analyte” as used herein is a broad term and is to be given its ordinary and customary meaning to a person of ordinary skill in the art and is not to be limited to a special or customized meaning. The term specifically may refer, without limitation, to the at least one analyte may be evaluated and/or detected quantitatively and/or qualitatively from the sensor signal, specifically the standard sensor signal or the economy sensor signal. The standard sensor signal may correlate with a concentration value of the analyte in the bodily fluid. Additionally or alternatively, the standard sensor signal may be proportional to the concentration value of the analyte in the bodily fluid. Additionally or alternatively, the concentration value of the analyte in the bodily fluid may be proportional to the determined current through the working electrode and the further electrode in the standard operation mode.

The analyte specifically may be selected from the group consisting of: glucose; lactate; or glutamate. The bodily fluid specifically may be selected from the group consisting of interstitial fluid, blood, blood plasma, urine and saliva. Thus, as an example, the analyte may be glucose, and the bodily fluid may be blood or may be interstitial fluid. Other embodiments, however, are feasible.

A consumption of the silver chloride of the redox material composition in the economy operation mode may be lower than or equal to a consumption of the silver chloride of the redox material composition in the standard operation mode, particularly under the conditions at which the standard sensor signal is equal to the threshold. The term “consumption” as used herein is a broad term and is to be given its ordinary and customary meaning to a person of ordinary skill in the art and is not to be limited to a special or customized meaning. The term specifically may refer, without limitation, to a use of a portion of a substance, specifically of a portion of the silver chloride, wherein the amount decreases due to this use. The amount of silver chloride may be consumed in the redox reaction occurring between the at least one analyte and the silver chloride.

In the standard operation mode, the detected value of the glucose may be indicated to the patient. In the economy operation mode, the glucose level as being too high may be indicated to the patient. The term “indicating” as used herein is a broad term and is to be given its ordinary and customary meaning to a person of ordinary skill in the art and is not to be limited to a special or customized meaning. The term specifically may refer, without limitation, to a detail or an information that is forwarded, particularly the patient. The information and/or the detail may be selected from at least one of: the detected value of the glucose; or the glucose level as being too high. The information and/or the detail may be forwarded visually, particularly by being displayed on a screen; and/or audibly; particularly by being played on a speaker.

The method may further comprise a step iii. According to the step iii., the method may comprise switching from the standard operation mode into the economy operation mode if, preferably in step ii., it is determined that the change of the operation mode of the analyte sensor from the standard operation mode into the economy operation mode is required, specifically if the standard sensor signal exceeds the threshold, wherein at least one economy sensor signal may be generated in the economy operation mode. The term “switching” as used herein is a broad term and is to be given its ordinary and customary meaning to a person of ordinary skill in the art and is not to be limited to a special or customized meaning. The term specifically may refer, without limitation, to a change of the operation mode of the analyte sensor, particularly from the standard operation to the economy operation.

The economy operation mode, as an example, may be performed either for a predetermined economy time span or until at least one condition for switching back from the economy operation mode into the standard operation mode is fulfilled. For checking the at least one condition, any measured economy sensor signal may be compared with the at least one economy threshold condition. Alternatively or in addition, an average of at least two measured economy sensor signals may be compared with the at least one economy threshold condition. The term “economy time span” as used herein is a broad term and is to be given its ordinary and customary meaning to a person of ordinary skill in the art and is not to be limited to a special or customized meaning. The term specifically may refer, without limitation, to an interval in time, particularly of at least 1 min; 2 min; 3 min; 4 min or 5 min. The term “condition for switching back” as used herein is a broad term and is to be given its ordinary and customary meaning to a person of ordinary skill in the art and is not to be limited to a special or customized meaning. The condition for switching back to the standard operation mode may be the economy sensor signal exceeding a threshold potential, specifically in the economy operation mode. The threshold potential may correspond to a threshold glucose level. The condition for switching back to the standard operation mode may be, alternatively or additionally, a falling of the evaluated glucose level below the threshold glucose level, specifically in the economy operation mode. The threshold glucose level may, again, be typically 200 mg/dl or 300 mg/dl.

At least one economy sensor signal may be generated in the economy operation mode by performing galvanostatic measurements for detecting the at least one analyte with the analyte sensor, wherein an current through the working electrode and the further electrode may be set to at least one predetermined economy current value, wherein an operating potential of the working electrode with respect to the further electrode may be determined, particularly wherein the predetermined economy current value may be kept constant during the determination of the operating potential.

The term “galvanostatic measurements” as used herein is a broad term and is to be given its ordinary and customary meaning to a person of ordinary skill in the art and is not to be limited to a special or customized meaning. The term specifically may refer, without limitation, to a measurement wherein the current through the electrodes, specifically the working electrode and the further electrode, is kept essentially constant, e.g., with a variation of less than 1%, less than 0.75% or even less than 0.5% tolerance, particularly during the monitoring of a plurality of sensor signals, specifically the monitoring of a plurality of economy sensor signals. The current may be kept constant at a predefined value during the generation of the plurality of sensor signals. The predefined value may depend on the sensor sensitivity. For example, the predefined value may be 25 nA for a threshold glucose level of 250 mg/dl and a sensor sensitivity of 0.1 nA/mg/dl. The predefined value may be 12.5 nA for a threshold glucose level of 250 mg/dl and a sensor sensitivity of 0.05 nA/mg/dl may. Thereby, the analyte sensor may be operated in a kinetic-controlled region, in which the economy sensor signal, particularly being the operating potential of the working electrode with respect to the further electrode, may particularly depend on the kinetics of the charge transfer. The operating potential in the economy mode may be regulated by the electronic unit, particularly to generate the predefined value of the current between the working electrode and the further electrode.

The term “economy sensor signal” as used herein is a broad term and is to be given its ordinary and customary meaning to a person of ordinary skill in the art and is not to be limited to a special or customized meaning. The term specifically may refer, without limitation, to the operating potential between the working electrode and the further electrode, particularly used to regulate the current between the working electrode and the further electrode. The potential may depend on the quantity of the at least one analyte, particularly the concentration value of the at least one analyte in the bodily fluid. Additionally or alternatively, the economy sensor signal may be derived from the operating potential, particularly by considering a calibration value and/or calibration function. Thereby, the economy sensor signal may be the glucose level.

The predetermined economy current value may be chosen not to exceed an electric threshold current through the working electrode and the further electrode occurring when the standard sensor signal equals the threshold. The term “not to exceed” as used herein is a broad term and is to be given its ordinary and customary meaning to a person of ordinary skill in the art and is not to be limited to a special or customized meaning. The term specifically may refer, without limitation, to the predetermined economy current value being at least one of lower than; or equal to the electric threshold current.

The economy sensor signal may comprise information about the at least one analyte, particularly wherein the economy sensor signal may be, specifically exclusively, analyzed for detecting the at least one analyte in the economy operation mode. The economy sensor signal may correlate with the concentration value of the analyte in the bodily fluid. Additionally or alternatively, the economy sensor signal may be a function of the concentration value of the analyte in the bodily fluid. Additionally or alternatively, the concentration value of the analyte in the bodily fluid may be a function of the operating potential of the working electrode with respect to the further electrode in the economy operation mode.

The economy may further comprise a step iv. According to the step iv., the method may comprise monitoring the economy sensor signal, when the analyte sensor is used in the economy operation mode. The economy may further comprise a step v. According to the step v., the method may comprise comparing the economy sensor signal with at least one economy threshold, specifically determining if the economy sensor signal falls below or exceeds the economy threshold, thereby determining if a change of an operation mode of the analyte sensor from the economy operation mode back into the standard operation mode is required.

The term “determining if the economy sensor signal falls below or exceeds the economy threshold” as used herein is a broad term and is to be given its ordinary and customary meaning to a person of ordinary skill in the art and is not to be limited to a special or customized meaning. The term specifically may refer, without limitation, to determining if the economy sensor signal, specifically when being the operating potential, exceeds the threshold potential, specifically in the economy operation mode. Alternatively or additionally, determining if the economy sensor signal, specifically when being the glucose level, falls below the threshold glucose level, specifically in the economy operation mode.

The economy may further comprise a step vi. According to the step vi., the method may comprise switching from the economy operation mode back into the standard operation mode if, preferably in step v., it is determined that the change of the operation mode of the analyte sensor from the economy operation mode back into the standard operation mode is required, specifically if the economy sensor signal falls below or exceeds the economy threshold.

The standard sensor signal and/or the economy sensor signal each may be selected from the group consisting of: electrical signals generated by the analyte sensor, specifically at least one of electrical current signals; electrical signals generated for regulating the analyte sensor; specifically electrical voltage signals; secondary signals derived from electrical signals generated by the analyte sensor or for regulating the analyte sensor, specifically concentration values of the analyte in the bodily fluid determined by using electrical signals generated by the analyte sensor or for regulating the analyte sensor. The electrical voltage signals may be generated by using the electronic unit. The term “concentration value” as used herein is a broad term and is to be given its ordinary and customary meaning to a person of ordinary skill in the art and is not to be limited to a special or customized meaning. The term specifically may refer, without limitation, to an item of information qualitatively or quantitatively indicating a concentration of at least one compound in at least one medium, e.g., a concentration by weight and/or by volume, such as the concentration of the analyte in the bodily fluid, specifically the abundance of the at least one analyte divided by the total volume of the bodily fluid. The concentration value may refer to at least one: mass concentration; molar concentration; number concentration; or volume concentration.

The method may further comprise deriving at least one concentration value of the analyte in the bodily fluid by using the standard sensor signal or the economy sensor signal, respectively, depending on a current operation mode of the analyte sensor. The term “deriving” as used herein is a broad term and is to be given its ordinary and customary meaning to a person of ordinary skill in the art and is not to be limited to a special or customized meaning. The term specifically may refer, without limitation, to determining the at least one concentration value by evaluating the respective sensor signal, particularly by further considering a calibration value and/or calibration function or transformation algorithm. Thereby, the at least one analyte may be detected.

The method may further comprise indicating to a user if the analyte sensor presently is operated in the standard operation mode or in the economy operation mode. The term “indicating” as used herein is a broad term and is to be given its ordinary and customary meaning to a person of ordinary skill in the art and is not to be limited to a special or customized meaning. The term specifically may refer, without limitation, to giving information to the user, specifically about the operation mode. The operation mode may be indicated by displaying an indication about the respective operation mode at which analyte sensor is operated to the user.

The method may comprise continuously detecting the analyte over a time span of at least one day, specifically over a time span of at least seven days. The term “time span” as used herein is a broad term and is to be given its ordinary and customary meaning to a person of ordinary skill in the art and is not to be limited to a special or customized meaning. The term specifically may refer, without limitation, to a time interval at which the analyte sensor is operated. During this time interval or time span the analyte sensor may detect continuously and/or reliably the at least one analyte, specifically with a sensitivity that is above a predetermined sensitivity, and particularly without an interruption for a maintenance procedure. Once the time span has elapsed, the analyte sensor may require a maintenance procedure or may be exchanged.

The method may comprise continuously detecting the analyte by at least one of permanently evaluating sensor signals of the analyte sensor and repeatedly evaluating sensor signals of the analyte sensor, specifically repeatedly evaluating sensor signals acquired in regular or irregular time intervals. The term “permanently evaluating” as used herein is a broad term and is to be given its ordinary and customary meaning to a person of ordinary skill in the art and is not to be limited to a special or customized meaning. The term specifically may refer, without limitation, to a continuous or repeated, specifically an uninterrupted, evaluating of the sensor signals, specifically for detecting the at least one analyte. The term “repeatedly evaluating” as used herein is a broad term and is to be given its ordinary and customary meaning to a person of ordinary skill in the art and is not to be limited to a special or customized meaning. The term specifically may refer, without limitation, to a recurring and/or repetitive and/or continual evaluating of the sensor signals, specifically for detecting the at least one analyte.

The method, as outlined above, essentially may refer to a method of operating the analyte sensor, such as an analyte sensor with the sensor-related features according to any one of the embodiments described above and/or according to any one of the embodiments described in further detail below. In other words, the method as proposed herein may be a method of operating an analyte sensor and/or an analyte sensor system as described in further detail below, for continuously in vivo detecting at least one analyte in a bodily fluid over a time span, the method using the at least one analyte sensor comprising the at least one working electrode, configured for performing the at least one electrochemical detection reaction with the analyte, and the at least one further electrode, the further electrode comprising the at least one redox material composition, the redox material composition comprising the silver and silver chloride, the method comprising the method steps as disclosed above, i.e., at least steps i. and ii., and optionally also one or more of the further method steps as discussed above.

The method of continuously in vivo detecting at least one analyte in a bodily fluid over a time span, specifically the method of operating the analyte sensor, may be at least partially computer-implemented, specifically steps i. and ii., and optionally also one or more or all of steps iii. to vi. The term “computer-implemented” as used herein is a broad term and is to be given its ordinary and customary meaning to a person of ordinary skill in the art and is not to be limited to a special or customized meaning. The term specifically may refer, without limitation, to the method being performed by at least one apparatus, specifically an analyte sensor system including a processor. The computer-implemented method may be implemented as at least one computer program that may be provided on a memory of the analyte sensor system.

In a second aspect of this disclosure, an analyte sensor system for in vivo continuously detecting at least one analyte in a bodily fluid over a measurement time span is disclosed. The analyte sensor system comprises:
a. at least one electrochemical analyte sensor comprising at least one working electrode, configured for performing at least one electrochemical detection reaction with the analyte, and at least one further electrode, the further electrode comprising at least one redox material composition, the redox material composition comprising silver and silver chloride;
b. a processor and a memory storing instructions which when executed cause the processor to perform at least the following steps:
   i. monitoring at least one standard sensor signal derived by using the analyte sensor in a standard operation mode, wherein, in the standard operation mode, potentiostatic measurements are performed for detecting the at least one analyte with the analyte sensor, wherein an potential of the working electrode with respect to the further electrode is set to at least one predetermined standard operating potential and wherein an current through the working electrode and the further electrode is determined, particularly wherein the predetermined standard operating potential is kept constant during the determination of the current; and
   ii. comparing the standard sensor signal with at least one threshold, specifically determining if the standard sensor signal exceeds the threshold, thereby determining if a change of an operation mode of the analyte sensor from the standard operation mode into an economy operation mode is required.

The term “processor” as used herein is a broad term and is to be given its ordinary and customary meaning to a person of ordinary skill in the art and is not to be limited to a special or customized meaning. The term specifically may refer, without limitation, to an arbitrary logic circuitry configured for performing basic operations of a computer or system, and/or, generally, to a device which is configured for performing calculations or logic operations. In particular, the processor may be configured for processing basic instructions that drive the computer or system. As an example, the processor may comprise at least one arithmetic logic unit (ALU), at least one floating-point unit (FPU), such as a math co-processor or a numeric co-processor, a plurality of registers, specifically registers configured for supplying operands to the ALU and storing results of operations, and a memory, such as an L1 and L2 cache memory. In particular, the processor may be a multi-core processor. Specifically, the processor may be or may comprise a central processing unit (CPU). Additionally or alternatively, the processor may be or may comprise a microprocessor, thus specifically the processor's elements may be contained in one single integrated circuitry (IC) chip. Additionally or alternatively, the processor may be or may comprise one or more application-specific integrated circuits (ASICs) and/or one or more field-programmable gate arrays (FPGAs) and/or one or more tensor processing unit (TPU) and/or one or more chip, such as a dedicated machine learning optimized chip, or the like. Additionally or alternatively, the processor may be or may comprise a micro controller unit (MCU). The micro controller unit may be coupled to an analog front end (AFE) or a digital potentiostat. The processor specifically may be configured, such as by software programming, for performing one or more evaluation operations. The term “memory” as used herein is a broad term and is to be given its ordinary and customary meaning to a person of ordinary skill in the art and is not to be limited to a special or customized meaning. The term specifically may refer, without limitation, to a unit used for storing computer readable information, specifically for providing the stored information to the processor.

In the economy operation mode, at least one economy sensor signal may be generated by performing galvanostatic measurements for detecting the at least one analyte with the analyte sensor, wherein an current through the working electrode and the further electrode is set to at least one predetermined economy current value, wherein an operating potential of the working electrode with respect to the further electrode is determined, particularly wherein the predetermined economy current value is kept constant during the determination of the operating potential.

The analyte sensor system may comprise an evaluation unit configured for analyzing the standard sensor signal, and optionally the economy sensor signal, for detecting the at least one analyte, particularly for determining at least one concentration value of the analyte in the bodily fluid. The term “evaluation unit” as used herein is a broad term, and is to be given its ordinary and customary meaning to a person of ordinary skill in the art, and is not to be limited to a special or customized meaning. The term specifically may refer, without limitation, to an arbitrary functional element configured for analyzing and/or processing data. The evaluation unit may specifically analyze and/or process measurement data, e.g., the measurement results as generated by the impedance measurement unit. The evaluation unit may in particular comprise at least one processor. The processor may specifically be configured, such as by software programming, for performing one or more evaluation operations on the measurement results. The evaluation unit, specifically the processor of the evaluation unit, may be part of the processor of feature b. as discussed above, e.g., by being fully or partially integrated into said processor. Alternatively, however, the evaluation unit may also be separate from the processor of feature b.

The analyte sensor system may be configured, e.g., by software programming, such as by corresponding instructions in the memory, for performing the method according to any one of the preceding claims referring to a method. Any definition of a term given in the context of the aspect and the embodiments related to the method apply accordingly to the aspect and the embodiments related to the analyte sensor system.

The further electrode may comprise the redox material composition in the form of at least one layer, specifically at least one layer deposited on at least one substrate. This layer may be a non-continuous layer, e.g., formed by a plurality of dots. The term “layer” as used herein is a broad term and is to be given its ordinary and customary meaning to a person of ordinary skill in the art and is not to be limited to a special or customized meaning. The term specifically may refer, without limitation, to an arbitrary aliquot of material forming a sheet or film, either as a standalone film or as a film deposited on a substrate. The layer may specifically be arranged in at least one bundle of a plurality of layers, such as in a stack and/or multilayered element, wherein at least one layer may be placed and/or laid upon or below at least one other layer.

The further electrode may comprise at least one layer comprising the redox material composition, particularly in a concentration of 80% to 100% of weight, specifically 90% of weight. The redox material composition may comprise silver in a concentration of 5% by weight to 20% by weight. The layer may further comprise a polymeric binder, particularly in a concentration of 5 to 20% of weight, specifically 10% of weight. This layer may be a non-continuous layer, e.g., formed by a plurality of dots.

The further electrode may be selected from the group consisting of: a counter electrode; a reference electrode; a combined counter/reference electrode. The term “combined counter/reference electrode” as used herein is a broad term and is to be given its ordinary and customary meaning to a person of ordinary skill in the art and is not to be limited to a special or customized meaning. The term specifically may refer, without limitation, to an electrode that is intended to be used as a counter electrode and a reference electrode, specifically offers the functionally of a counter electrode and a reference electrode.

The further electrode may be at least partially coated by an insulating material. The term “insulating material” as used herein is a broad term and is to be given its ordinary and customary meaning to a person of ordinary skill in the art and is not to be limited to a special or customized meaning. The term specifically may refer, without limitation, to at least one electrically insulating material, especially in order to avoid unwanted currents between electrically conducting elements. By way of example, the electrically insulating material may be selected from the group consisting of polyethylene terephthalate (PET) and polycarbonate (PC). However, other kinds of electrically insulating materials may also be feasible.

The insulating material may leave open at least one window through which the further electrode is accessible for the analyte. The term “window” as used herein is a broad term and is to be given its ordinary and customary meaning to a person of ordinary skill in the art and is not to be limited to a special or customized meaning. The term specifically may refer, without limitation, to at least one area of the insulating material that leaves an opening so that the further electrode, particularly the at least one layer comprising the redox material composition, is not covered by the insulating material, specifically so that the further electrode, particularly the at least one layer comprising the redox material composition, is in contact with the bodily fluid in such a way that an redox reaction may occur between the at least one analyte and the further electrode.

The window specifically may have an open area of 0.1% to 0.5% of an overall electrode area of the further electrode. The area of the at least one window may be smaller than the area of the layer comprising the redox material composition, specifically covered by the insulating material. The insulating material may cover, specifically partially or fully, the layer comprising the redox material composition, so that only the part of the layer comprising the redox material composition that is left open by the window is accessible for the analyte. The full area build by the at least one window may be filled with the layer comprising the redox material composition. The total area may be smaller than 0.1 mm²; 0.09 mm²; 0.08 mm²; or 0.07 mm².

The electrochemical analyte sensor specifically may comprise at least one substrate, wherein the working electrode and the further electrode are deposited on the substrate. The term “substrate” as used herein is a broad term and is to be given its ordinary and customary meaning to a person of ordinary skill in the art and is not to be limited to a special or customized meaning. The term specifically may refer, without limitation, to an arbitrary element designed to carry one or more other elements fully or partially disposed thereon and/or therein. Particularly, the substrate may be a planar substrate. The substrate may, specifically, have an elongated shape, such as a strip shape or a bar shape; however, other kinds of shapes may also be feasible. The working electrode and the further electrode may be deposited on opposing sides of the substrate. The substrate may comprises a flexible substrate, specifically having a strip shape or a rod shape.

The electrochemical analyte sensor specifically may be configured for at least partially being transcutaneously inserted into a body tissue. The term “transcutaneously” as used herein is a broad term and is to be given its ordinary and customary meaning to a person of ordinary skill in the art and is not to be limited to a special or customized meaning. The term specifically may refer, without limitation, to penetrating and/or entering and/or passing through the skin of the user or patient.

The analyte sensor specifically may comprise at least one implantable part for transcutaneous insertion into the body tissue and at least one contacting portion comprising at least one electrode pad for electrically contacting the working electrode and at least one contacting pad for electrically contacting the further electrode. The term “implantable part” as used herein is a broad term and is to be given its ordinary and customary meaning to a person of ordinary skill in the art and is not to be limited to a special or customized meaning. The term specifically may refer, without limitation, to a part or component of an element configured to be insertable into an arbitrary body tissue, specifically the working electrode. Other parts or components of the analyte sensor may remain outside of the body tissue, e.g., counter electrode and/or reference electrode or combined counter/reference electrode may remain outside of the body tissue. Preferably, the insertable portion may fully or partially comprise a biocompatible surface, which may have as little detrimental effects on the user or the body tissue as possible, at least during typical durations of use. For this purpose, the insertable portion may be fully or partially covered with at least one biocompatibility membrane layer, such as at least one polymer membrane, for example, a gel membrane.

The working electrode may comprise at least one detection material, wherein the detection material comprises at least one enzyme configured for performing an analyte specific reaction with the analyte to be detected.

The enzyme specifically may be selected from the group consisting of glucose oxidase, glucose dehydrogenase, lactate oxidase, lactate dehydrogenase, glutamate oxidase and glutamate dehydrogenase. Other enzymes, however, are also feasible, as, e.g., discussed above.

The electrochemical analyte sensor specifically may comprise at least one membrane, the membrane being permeable for the analyte to be detected, specifically the membrane being impermeable for materials of the working electrode and the further electrode, the membrane at least partially surrounding the working electrode and the further electrode. The term “being permeable for the analyte to be detected” as used herein is a broad term and is to be given its ordinary and customary meaning to a person of ordinary skill in the art and is not to be limited to a special or customized meaning. The term specifically may refer, without limitation, to the membrane allowing certain molecules or ions to pass through it by diffusion, specifically allowing the analyte to be detected to pass through it. The membrane may limit the diffusion of the analyte onto the layer comprising the redox material composition. The term “impermeable for materials of the working electrode and the further electrode” as used herein is a broad term and is to be given its ordinary and customary meaning to a person of ordinary skill in the art and is not to be limited to a special or customized meaning. The term specifically may refer, without limitation, to the membrane not allowing certain molecules or ions to pass through it by diffusion, specifically not allowing products of a redox reaction occurring at the respective electrode to pass through it.

In a third aspect of this disclosure, a computer program comprising instructions is disclosed, which, when the program is executed by the processor of the analyte sensor system according to any one of the preceding claims referring to an analyte sensor system, cause the analyte sensor system to perform the method according to any one of the preceding claims referring to a method, specifically at least method steps i. and ii., and optionally also one or more of steps iii., iv., v. and vi. The term “computer program” as used herein is a broad term and is to be given its ordinary and customary meaning to a person of ordinary skill in the art and is not to be limited to a special or customized meaning. The term specifically may refer, without limitation, to at least one executable instruction for at least one programmable apparatus, specifically a computer, preferably a sequence of executable instructions, for processing and/or solving of at least one function and/or at least one task and/or at least one problem by using at least one programmable apparatus, specifically a computer, preferably for performing some or all steps of any one of the methods according to any aspect or embodiment as described within this disclosure.

The computer program may further be used for evaluating the data for detecting the at least one analyte, this evaluation may depend on the mode of operation of the analyte sensor, particularly by evaluating the current or the potential between the working electrode and the further electrode.

In a fourth aspect of this disclosure, a computer-readable storage medium comprising instructions is disclosed, which, when the instructions are executed by the processor of the analyte sensor system according to any one of the preceding aspects or embodiments, specifically referring to the method and/or referring to the analyte sensor system, cause the analyte sensor system to perform the method according to any one of the preceding claims referring to a method, specifically at least method steps i. and ii., and optionally also one or more of steps iii., iv., v. and vi. As used herein, the term “computer-readable storage medium” specifically may refer to a non-transitory data storage means, such as a hardware storage medium having stored thereon computer-executable instructions. The computer-readable data carrier or storage medium specifically may be or may comprise a storage medium such as a random-access memory (RAM) and/or a read-only memory (ROM).

Thus, specifically, one, more than one or even all of method steps i. to vi., more specifically steps i. and ii., and optionally also one or more or all of steps iii. to vi., as indicated above may be performed by using a computer or a computer network, preferably by using a computer program.

Further disclosed and proposed herein is a computer program product having program code means, in order to perform the method according to this disclosure in one or more of the embodiments enclosed herein when the program is executed on a computer or computer network. Specifically, the program code means may be stored on a computer-readable data carrier and/or on a computer-readable storage medium.

Further disclosed and proposed herein is a data carrier having a data structure stored thereon, which, after loading into a computer or computer network, such as into a working memory or main memory of the computer or computer network, may execute the method according to one or more of the embodiments disclosed herein.

Further disclosed and proposed herein is a non-transient computer-readable medium including instructions that, when executed by one or more processors, cause the one or more processors to perform a method of continuously in vivo detecting at least one analyte in a bodily fluid over a time span as described in the aspect and the embodiments, specifically at least method steps i. and ii., and optionally also one or more of steps iii., iv., v. and vi.

Further disclosed and proposed herein is a computer program product with program code means stored on a machine-readable carrier, in order to perform the method according to one or more of the embodiments disclosed herein, when the program is executed on a computer or computer network. As used herein, a computer program product refers to the program as a tradable product. The product may generally exist in an arbitrary format, such as in a paper format, or on a computer-readable data carrier and/or on a computer-readable storage medium. Specifically, the computer program product may be distributed over a data network.

Finally, disclosed and proposed herein is a modulated data signal which contains instructions readable by a computer system or computer network, for performing the method according to one or more of the embodiments disclosed herein.

Referring to the computer-implemented aspects of this disclosure, one or more of the method steps or even all of the method steps of the method according to one or more of the embodiments disclosed herein may be performed by using a computer or computer network. Thus, generally, any of the method steps including provision and/or manipulation of data may be performed by using a computer or computer network. Generally, these method steps may include any of the method steps, typically except for method steps requiring manual work, such as providing the samples and/or certain aspects of performing the actual measurements.

Specifically, further disclosed herein are:
a computer or computer network comprising at least one processor, wherein the processor is adapted to perform the method according to one of the embodiments described in this description, specifically at least method steps i. and ii., and optionally also one or more of steps iii., iv., v. and vi.,
a computer loadable data structure that is adapted to perform the method according to one of the embodiments described in this description while the data structure is being executed on a computer, specifically at least method steps i. and ii., and optionally also one or more of steps iii., iv., v. and vi.,
a computer program, wherein the computer program is adapted to perform the method according to one of the embodiments described in this description while the program is being executed on a computer, specifically at least method steps i. and ii., and optionally also one or more of steps iii., iv., v. and vi.,
a computer program comprising program means for performing the method according to one of the embodiments described in this description while the computer program is being executed on a computer or on a computer network, specifically at least method steps i. and ii., and optionally also one or more of steps iii., iv., v. and vi.,
a computer program comprising program means according to the preceding embodiment, wherein the program means are stored on a storage medium readable to a computer, specifically at least method steps i. and ii., and optionally also one or more of steps iii., iv., v. and vi.,
a storage medium, wherein a data structure is stored on the storage medium and wherein the data structure is adapted to perform the method according to one of the embodiments described in this description after having been loaded into a main and/or working storage of a computer or of a computer network, specifically at least method steps i. and ii., and optionally also one or more of steps iii., iv., v. and vi., and
a computer program product having program code means, wherein the program code means can be stored or are stored on a storage medium, for performing the method according to one of the embodiments described in this description, if the program code means are executed on a computer or on a computer network, specifically at least method steps i. and ii., and optionally also one or more of steps iii., iv., v. and vi.
   In the context of this paragraph, an embodiment may refer to the aspect and/or a preferred embodiment of the method as described above.

The method of continuously in vivo detecting at least one analyte in a bodily fluid over a time span, the analyte sensor system for in vivo continuously detecting at least one analyte in a bodily fluid over a measurement time span, the computer program and the computer-readable storage medium presents a variety of advantages over prior art.

By changing the operation mode, particularly between the standard operation mode and the economy operation mode, or vice versa, the consumption of the redox material composition may be decreased, specifically when compared to a conventional driving scheme of an analyte detector that is operated without changing of the operation mode. Thereby, specifically the consumption of silver chloride may be decreased, as the current through the further electrode and the working electrode may be reduced. This may prevent excessive use, particularly in time periods during which the actual glucose level is particularly high, as might it be the case during a hyperglycemic period.

In case the glucose level may be particularly high, the exact value of the glucose level may not be of particular importance. Therefore, some clinically relevant maximum glucose value may be defined, or specifically a threshold glucose level may be defined. In the economy operation mode that may be used for operating the analyte sensor in case the determined glucose level is above the threshold glucose level, the current may be regulated in such a manner that it is kept constant at a defined value. Thereby, the maximum consumption of silver chloride may be capped. The maximum consumption of the silver chloride may, thus, be well defined, particularly for a worst case scenario. This may make it easy to calculate a reliable silver chloride content of the analyte sensor.

As a result, the analyte sensor may require only a reduced amount of silver chloride compared to analyte sensors having a conventional driving mode, and that particularly may not be operated in an economy operation mode. Thereby, the bio-compatibility of the analyte sensor may be enhanced, as an immune response caused by the silver chloride may be reduced, specifically due to the reduced amount of silver chloride in the analyte sensor. The efficiency or sensitivity of the analyte sensor may further be increased as the reduced immune response may interfere less with the produced sensor signal. Additionally or alternatively, reducing the amount of redox material comprising silver may allow for minimizing the sensor size.

By coating at least a part of the layer comprising the redox material composition by using insulating material, a portion of the silver chloride may be hidden. Therefore, the immune response is further reduced. The use of the membrane may have the same effect.

Summarizing and without excluding further possible embodiments, the following embodiments may be envisaged:

Embodiment 1: A method of continuously in vivo detecting at least one analyte in a bodily fluid over a time span, the method using at least one analyte sensor comprising at least one working electrode, configured for performing at least one electrochemical detection reaction with the analyte, and at least one further electrode, the further electrode comprising at least one redox material composition, the redox material composition comprising silver and silver chloride, the method comprising the following steps:
i. monitoring at least one standard sensor signal derived by using the analyte sensor in a standard operation mode, wherein, in the standard operation mode, potentiostatic measurements are performed for detecting the at least one analyte with the analyte sensor, wherein a potential of the working electrode with respect to the further electrode is set to at least one predetermined standard operating potential and wherein an current through the working electrode and the further electrode is determined, particularly wherein the predetermined standard operating potential is kept constant during the determination of the current; and
ii. comparing the standard sensor signal with at least one threshold, specifically for determining if the standard sensor signal exceeds the threshold, thereby determining if a change of an operation mode of the analyte sensor from the standard operation mode into an economy operation mode is required.

Embodiment 2: The method according to the preceding embodiment, wherein the standard sensor signal comprises information about the at least one analyte, particularly wherein the standard sensor signal is, specifically exclusively, analyzed for detecting the at least one analyte in the standard operation mode.

Embodiment 3: The method according to any one of the preceding embodiments, wherein the standard sensor signal correlates with an concentration value of the analyte in the bodily fluid, particularly wherein the standard sensor signal is proportional to the concentration value of the analyte in the bodily fluid, more particularly wherein the concentration value of the analyte in the bodily fluid is proportional to the determined current through the working electrode and the further electrode in the standard operation mode.

Embodiment 4: The method according to any one of the preceding embodiments, wherein the analyte is selected from at least one of:
glucose;
lactate; or
glutamate.

Embodiment 5: The method according to any one of the preceding embodiments, wherein the bodily fluid is selected from the group consisting of interstitial fluid, blood, blood plasma, urine and saliva.

Embodiment 6: The method according to any one of the preceding embodiments, wherein, in the economy operation mode, a consumption of the silver chloride of the redox material composition is lower than or equal to a consumption of the silver chloride of the redox material composition in the standard operation mode, particularly under the conditions at which the standard sensor signal is equal to the threshold.

Embodiment 7: The method according to any one of the preceding embodiments, further comprising:
iii. switching from the standard operation mode into the economy operation mode if, in step ii., it is determined that the change of the operation mode of the analyte sensor from the standard operation mode into the economy operation mode is required, specifically if the standard sensor signal exceeds the threshold, wherein at least one economy sensor signal is generated in the economy operation mode.

Embodiment 8: The method according to the preceding embodiment, wherein the economy operation mode is performed either for a predetermined economy time span or until at least one condition for switching back from the economy operation mode into the standard operation mode is fulfilled.

Embodiment 9: The method according to any one of the preceding embodiments, wherein, in the economy operation mode, at least one economy sensor signal is generated by performing galvanostatic measurements for detecting the at least one analyte with the analyte sensor, wherein an current through the working electrode and the further electrode is set to at least one predetermined economy current value, wherein an operating potential of the working electrode with respect to the further electrode is determined, particularly wherein the predetermined economy current value is kept constant during the determination of the operating potential.

Embodiment 10: The method according to the preceding embodiment, wherein the predetermined economy current value is chosen not to exceed an electric threshold current through the working electrode and the further electrode occurring when the standard sensor signal equals the threshold.

Embodiment 11: The method according to any one of the four preceding embodiments, wherein the economy sensor signal comprises information about the at least one analyte, particularly wherein the economy sensor signal is, specifically exclusively, analyzed for detecting the at least one analyte in the economy operation mode.

Embodiment 12: The method according to any one of the five preceding embodiments, wherein the economy sensor signal correlates with the concentration value of the analyte in the bodily fluid, particularly wherein the economy sensor signal is a function of the concentration value of the analyte in the bodily fluid, more particularly wherein the concentration value of the analyte in the bodily fluid is a function of the operating potential of the working electrode with respect to the further electrode in the economy operation mode.

Embodiment 13: The method according to any one of the six preceding embodiments, further comprising:
iv. monitoring, when the analyte sensor is used in the economy operation mode, the economy sensor signal; and
v. comparing the economy sensor signal with at least one economy threshold, thereby determining if a change of an operation mode of the analyte sensor from the economy operation mode back into the standard operation mode is required.

Embodiment 14: The method according to the preceding embodiment, further comprising:
vi. switching from the economy operation mode back into the standard operation mode if, in step v., it is determined that the change of the operation mode of the analyte sensor from the economy operation mode back into the standard operation mode is required.

Embodiment 15: The method according to any one of the preceding embodiments, wherein the standard sensor signal and the economy sensor signal each are selected from the group consisting of: electrical signals generated by the analyte sensor, specifically at least one electrical current signals; electrical signals generated for regulating the analyte sensor, specifically at least one electrical voltage signal; secondary signals derived from electrical signals generated by the analyte sensor or for regulating the analyte sensor, specifically concentration values of the analyte in the bodily fluid determined by using electrical signals generated by the analyte sensor or for regulating the analyte sensor.

Embodiment 16: The method according to any one of the preceding embodiments, wherein the method further comprises deriving at least one concentration value of the analyte in the bodily fluid by using the standard sensor signal or the economy sensor signal, respectively, depending on a current operation mode of the analyte sensor.

Embodiment 17: The method according to any one of the preceding embodiments, wherein the method further comprises indicating to a user if the analyte sensor presently is operated in the standard operation mode or in the economy operation mode.

Embodiment 18: The method according to any one of the preceding embodiments, wherein the method comprises continuously detecting the analyte over a time span of at least one day, specifically over a time span of at least seven days.

Embodiment 19: The method according to any one of the preceding embodiments, wherein the method comprises continuously detecting the analyte by at least one of permanently evaluating sensor signals of the analyte sensor and repeatedly evaluating sensor signals of the analyte sensor, specifically repeatedly evaluating sensor signals acquired in regular or irregular time intervals.

Embodiment 20: The method according to any one of the preceding method embodiments, wherein the method is at least partially computer-implemented, specifically steps i. and ii., and optionally also one or more or all of steps iii. to vi.

Embodiment 21: An analyte sensor system for in vivo continuously detecting at least one analyte in a bodily fluid over a measurement time span, the analyte sensor system comprising:
a. at least one electrochemical analyte sensor comprising at least one working electrode, configured for performing at least one electrochemical detection reaction with the analyte, and at least one further electrode, the further electrode comprising at least one redox material composition, the redox material composition comprising silver and silver chloride;
b. a processor and a memory storing instructions which when executed cause the processor to perform at least the following steps:
   i. monitoring at least one standard sensor signal derived by using the analyte sensor in a standard operation mode, wherein, in the standard operation mode, potentiostatic measurements are performed for detecting the at least one analyte with the analyte sensor, wherein an potential of the working electrode with respect to the further electrode is set to at least one predetermined standard operating potential and wherein an current through the working electrode and the further electrode is determined, particularly wherein the predetermined standard operating potential is kept constant during the determination of the current; and
   ii. comparing the standard sensor signal with at least one threshold, specifically determining if the standard sensor signal exceeds the threshold, thereby determining if a change of an operation mode of the analyte sensor from the standard operation mode into an economy operation mode is required.

Embodiment 22: The analyte sensor system according to the preceding embodiment, wherein, in the economy operation mode, at least one economy sensor signal is generated by performing galvanostatic measurements for detecting the at least one analyte with the analyte sensor, wherein an current through the working electrode and the further electrode is set to at least one predetermined economy current value, wherein an operating potential of the working electrode with respect to the further electrode is determined, particularly wherein the predetermined economy current value is kept constant during the determination of the operating potential.

Embodiment 23: The analyte sensor system according to any one of the preceding embodiments referring to an analyte sensor system, wherein the analyte sensor system comprises an evaluation unit configured for analyzing the standard sensor signal, and optionally the economy sensor signal, for detecting the at least one analyte, particularly for determining at least one concentration value of the analyte in the bodily fluid.

Embodiment 24: The analyte sensor system according to any one of the preceding embodiments referring to an analyte sensor system, wherein the analyte sensor system is configured for performing the method according to any one of the preceding embodiments referring to a method.

Embodiment 25: The analyte sensor system according to any one of the preceding embodiments referring to an analyte sensor system, wherein the further electrode comprises the redox material composition in the form of at least one layer, specifically at least one layer deposited on at least one substrate.

Embodiment 26: The analyte sensor system according to any one of the preceding embodiments referring to an analyte sensor system, wherein the further electrode is selected from the group consisting of: a counter electrode; a reference electrode; a combined counter/reference electrode.

Embodiment 27: The analyte sensor system according to any one of the preceding embodiments referring to an analyte sensor system, wherein the further electrode comprises at least one layer comprising the redox material composition.

Embodiment 28: The analyte sensor system according to any one of the preceding embodiments referring to an analyte sensor system, wherein the redox material composition comprises silver in a concentration of 5% by weight to 20% by weight.

Embodiment 29: The analyte sensor system according to any one of the preceding embodiments referring to and analyte sensor system, wherein the further electrode is at least partially coated by an insulating material.

Embodiment 30: The analyte sensor system according to the preceding embodiment, wherein the insulating material leaves open at least one window through which the further electrode is accessible for the analyte.

Embodiment 31: The analyte sensor system according to the preceding embodiment, wherein the window has an open area of 0.1% to 0.5% of and overall electrode area of the further electrode.

Embodiment 32: The analyte sensor system according to any one of the preceding embodiments referring to an analyte sensor system, wherein the electrochemical analyte sensor comprises at least one substrate, wherein the working electrode and the further electrode are deposited on the substrate.

Embodiment 33: The analyte sensor system according to the preceding embodiment, wherein the working electrode and the further electrode are deposited on opposing sides of the substrate.

Embodiment 34: The analyte sensor system according to any one of the two preceding embodiments, wherein the substrate comprises a flexible substrate, specifically having a strip shape or a rod shape.

Embodiment 35: The analyte sensor system according to any one of the preceding embodiments, wherein the electrochemical analyte sensor is configured for at least partially being transcutaneously inserted into a body tissue.

Embodiment 36: The analyte sensor system according to the preceding embodiment, wherein the analyte sensor comprises at least one implantable part for transcutaneous insertion into the body tissue and at least one contacting portion comprising at least one electrode pad for electrically contacting the working electrode and at least one contacting pad for electrically contacting the further electrode.

Embodiment 37: The analyte sensor system according to any one of the preceding embodiments referring to an analyte sensor system, wherein the working electrode comprises at least one detection material, wherein the detection material comprises at least one enzyme configured for performing an analyte specific reaction with the analyte to be detected.

Embodiment 38: The analyte sensor system according to the preceding embodiment, wherein the enzyme is selected from the group consisting of glucose oxidase, glucose dehydrogenase, lactate oxidase, lactate dehydrogenase, glutamate oxidase and glutamate dehydrogenase.

Embodiment 39: The analyte sensor system according to any one of the preceding embodiments referring to an analyte sensor system, wherein the electrochemical analyte sensor comprises at least one membrane, the membrane being permeable for the analyte to be detected, specifically the membrane being impermeable for materials of the working electrode and the further electrode, the membrane at least partially surrounding the working electrode and the further electrode.

Embodiment 40: A computer program comprising instructions which, when the program is executed by the processor of the analyte sensor system according to any one of the preceding embodiments referring to an analyte sensor system, cause the analyte sensor system to perform the method according to any one of the preceding embodiments referring to a method.

Embodiment 41: A computer-readable storage medium comprising instructions which, when the instructions are executed by the processor of the analyte sensor system according to any one of the preceding embodiments referring to an analyte sensor system, cause the analyte sensor system to perform the method according to any one of the preceding embodiments referring to a method.

### BRIEF DESCRIPTION OF THE DRAWINGS

The above-mentioned aspects of exemplary embodiments will become more apparent and will be better understood by reference to the following description of the embodiments taken in conjunction with the accompanying drawings, wherein:

FIG. 1 shows an exemplary embodiment of an analyte sensor system;

FIG. 2 shows an exemplary embodiment of a method of continuously in vivo detecting at least one analyte in a bodily fluid over a time span;

FIG. 3 shows a current-voltage characteristic of an exemplarily embodiment of an analyte sensor; and

FIG. 4 shows an error grid analysis indicating the required sensitivity of an analyte sensor system depending on a reference blood glucose level.

### DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of this disclosure.

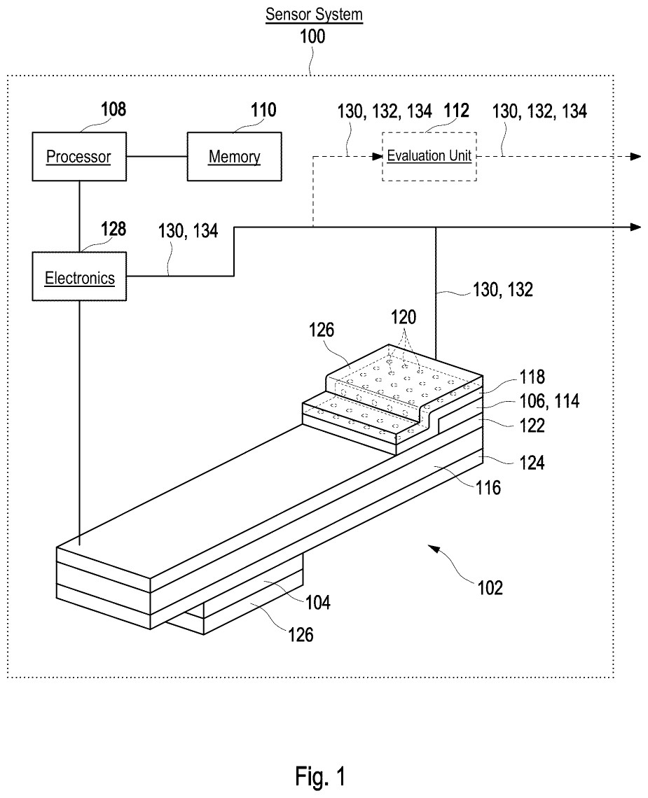

In FIG. 1, an exemplary embodiment of an analyte sensor system 100 is depicted in a schematic view. The analyte sensor system 100 is designated for in vivo continuously detecting at least one analyte in a bodily fluid over a measurement time span. The analyte specifically may be selected from the group consisting of glucose, lactate, and glutamate. Additionally or alternatively, one or more other analytes may be selected. The bodily fluid may be interstitial fluid, blood, blood plasma, urine, and/or saliva. There may be further options for the bodily fluid.

The analyte sensor system 100 comprises at least one electrochemical analyte sensor 102 that comprises at least one working electrode 104, configured for performing at least one electrochemical detection reaction with the analyte, and at least one further electrode 106. The further electrode 106 comprises at least one redox material composition and the redox material composition comprises silver and silver chloride.

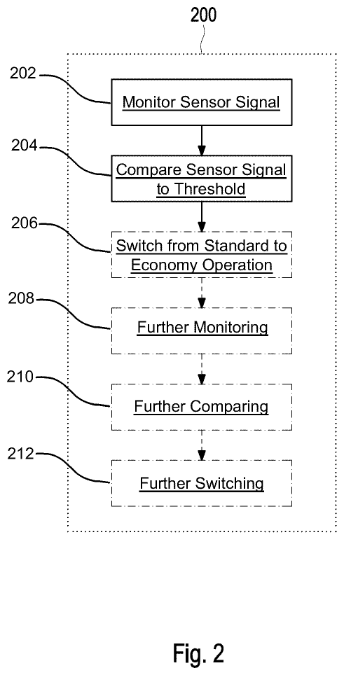

The analyte sensor system 100 further comprises a processor 108 and a memory 110. A computer program comprising instructions which, when the program is executed by the processor 108 of the analyte sensor system 100 may cause the analyte sensor system 100 to perform a method 200 of continuously in vivo detecting at least one analyte in a bodily fluid over a time span. The instructions may be stored in the memory 110. The method 200 is described in more detail in FIG. 2. Alternatively, a computer-readable storage medium may be provided comprising instructions which, when the instructions are executed by the processor 108 of the analyte sensor system 100 cause the analyte sensor system 100 to perform the method 200. To perform the method 200, the processor 108 controls and/or regulates an electronic unit 128 that controls and/or regulates the analyte sensor 102, particularly the voltage between the working electrode 104 and the further electrode 106.

The analyte sensor system 100 may further comprise an evaluation unit 112 configured for analyzing a sensor signal 130 of the analyte sensor 102 and/or the electronic unit 128 for detecting the at least one analyte, particularly for determining at least one concentration value of the analyte in the bodily fluid. The sensor signal 130 may be the current between the working electrode 104 and the further electrode 106 and/or the voltage between the working electrode 104 and the further electrode 106. The sensor signal 130 may further be the glucose level derived from the current and/or the voltage, particularly by using a calibration value and/or function, specifically by using the evaluation unit 112.

The electrochemical analyte sensor 102 is configured for at least partially being transcutaneously inserted into a body tissue. The analyte sensor 102 may comprise at least one implantable part for a transcutaneous insertion into the body tissue and at least one contacting portion comprising at least one electrode pad 122 for electrically contacting the further electrode 106 and at least one contacting pad 124 for electrically contacting the working electrode 104.

The electrochemical analyte sensor 102 may comprise at least one substrate 116, wherein the working electrode 104 and the further electrode 106 may be deposited on the substrate 116, particularly on opposite sides of the substrate 116. The contacting pad 124 may be arranged between the working electrode 104 and the substrate 116. The electrode pad 122 may be arranged between the further electrode 106 and the substrate 116. The substrate 116, as an example, may be strip-shaped. Alternatively, as an example, the substrate 116 may have a rod shape. The substrate 116 may be a flexible substrate 116.

The electrochemical analyte sensor 102 may further comprise at least one membrane 126. The membrane 126 may be permeable for the analyte to be detected. Specifically, the membrane 126 may be impermeable for materials of the working electrode 104 and the further electrode 106, specifically for materials produced in a redox reaction at the respective electrode. The membrane 126 may at least partially surround the working electrode 104 and the further electrode 106. Alternatively, the membrane 126 may at least partially surround the working electrode 104 or the further electrode 106.

The further electrode 106 comprises the redox material composition in the form of at least one layer 114, specifically at least one layer 114 deposited on the at least one substrate 116. The further electrode 106, thereby, may comprise at least one layer 114 comprising the redox material composition. The redox material composition, specifically, may comprise silver in a concentration of 5% by weight to 20% by weight.

The further electrode 106 may at least partially be coated by an insulating material 118. The insulating material 118 may leave open at least one window 120 through which the further electrode 106 may be accessible for the analyte. The at least one window 120, specifically, may have an open area of 0.1% to 0.5% of an overall electrode area of the further electrode 106. The further electrode 106 specifically may be selected from the group consisting of: a counter electrode; a reference electrode; a combined counter/reference electrode.

The working electrode 104 may comprise at least one detection material, wherein the detection material may comprise at least one enzyme configured for performing an analyte specific reaction with the analyte to be detected. The enzyme, specifically, may be selected from the group consisting of glucose oxidase, glucose dehydrogenase, lactate oxidase, lactate dehydrogenase, glutamate oxidase and glutamate dehydrogenase.

In FIG. 2, a flow chart of an embodiment of the method 200 of continuously in vivo detecting at least one analyte in a bodily fluid over a time span is depicted. According to the exemplary embodiment of FIG. 2 all steps of the method 200 may be computer-implemented. Alternatively, the method may be partially computer-implemented. Thus, specifically, steps i. and ii. may be computer-implemented, and additionally also one or more or all of steps iii. to vi. may be computer-implemented.

The method 200 makes use of the analyte sensor 102 as discussed above. Thus, the method 200 may be a method for operating the analyte sensor 102 and/or for operating the analyte sensor system 100. The analyte sensor 102, as outlined above, comprises the at least one working electrode 104, configured for performing at least one electrochemical detection reaction with the analyte, and the at least one further electrode 106. The method 200 comprises the following steps:
i. a monitoring step 202, wherein at least one standard sensor signal 132 derived by using the analyte sensor 102 in a standard operation mode is monitored, wherein, in the standard operation mode, potentiostatic measurements are performed for detecting the at least one analyte with the analyte sensor 102, wherein a potential of the working electrode 104 with respect to the further electrode 106 is set to at least one predetermined standard operating potential and wherein an current through the working electrode 104 and the further electrode 106 is determined, particularly wherein the predetermined standard operating potential is kept constant during the determination of the current; and
ii. a comparing step 204, wherein the standard sensor signal 132 with at least one threshold, specifically for determining if the standard sensor signal 132 exceeds the threshold is compared, thereby determining if a change of an operation mode of the analyte sensor 102 from the standard operation mode into an economy operation mode is required.

The standard sensor signal 132 may comprise information about the at least one analyte, particularly wherein the standard sensor signal 132 is, specifically exclusively, analyzed for detecting the at least one analyte in the standard operation mode. The standard sensor signal 132 may correlate with a concentration value of the analyte in the bodily fluid, particularly wherein the standard sensor signal 132 is proportional to the concentration value of the analyte in the bodily fluid, more particularly wherein the concentration value of the analyte in the bodily fluid is proportional to the determined current through the working electrode 104 and the further electrode 106 in the standard operation mode.

In the economy operation mode, a consumption of the silver chloride of the redox material composition may be lower than or equal to a consumption of the silver chloride of the redox material composition in the standard operation mode, particularly under the conditions at which the standard sensor signal 132 is equal to the threshold.

The method 200 may further comprise:
iii. a switching step 206, in which it is switched from the standard operation mode into the economy operation mode if, in the comparing step 204, it is determined that the change of the operation mode of the analyte sensor 102 from the standard operation mode into the economy operation mode is required, specifically if the standard sensor signal 132 exceeds the threshold.

The economy operation mode may be performed either for a predetermined economy time span or until at least one condition for switching back from the economy operation mode into the standard operation mode is fulfilled.

In the economy operation mode, at least one economy sensor signal 134 may be generated by performing galvanostatic measurements for detecting the at least one analyte with the analyte sensor 102, wherein an current through the working electrode 104 and the further electrode 106 may be set to at least one predetermined economy current value, wherein an operating potential of the working electrode 104 with respect to the further electrode 106 may be determined, particularly wherein the predetermined economy current value may be kept constant during the determination of the operating potential. The predetermined economy current value may be chosen not to exceed an electric threshold current through the working electrode 104 and the further electrode 106 occurring when the standard sensor signal 132 equals the threshold.

The economy sensor signal 134 may comprise information about the at least one analyte, particularly wherein the economy sensor signal 134 is, specifically exclusively, analyzed for detecting the at least one analyte in the economy operation mode. The economy sensor signal 134 may correlate with the concentration value of the analyte in the bodily fluid, particularly wherein the economy sensor signal 134 may be a function of the concentration value of the analyte in the bodily fluid, more particularly wherein the concentration value of the analyte in the bodily fluid may be a function of the operating potential of the working electrode 104 with respect to the further electrode 106 in the economy operation mode.

The method 200 may further comprise:
vii. a further monitoring step 208, when the analyte sensor 102 is used in the economy operation mode, wherein the economy sensor signal 134 is monitored; and
viii. a further comparing step 210, wherein the economy sensor signal 134 is compared with at least one economy threshold, thereby determining if a change of an operation mode of the analyte sensor 102 from the economy operation mode back into the standard operation mode is required.

The method 200 may further comprise:
ix. a further switching step 212, wherein it is switched from the economy operation mode back into the standard operation mode if, in the further comparing step 210, it is determined that the change of the operation mode of the analyte sensor 102 from the economy operation mode back into the standard operation mode is required.

The standard sensor signal 132 and the economy sensor signal 134 each may be selected from the group consisting of: electrical signals generated by the analyte sensor 102, specifically at least one of electrical current signals; electrical signals generated for regulating the analyte sensor 102; specifically electrical voltage signals; secondary signals derived from electrical signals generated by the analyte sensor 102, specifically concentration values of the analyte in the bodily fluid determined by using electrical signals generated by the analyte sensor 102.

The method 200 further may comprise deriving at least one concentration value of the analyte in the bodily fluid by using the standard sensor signal 132 or the economy sensor signal 134, respectively, depending on a current operation mode of the analyte sensor 102. The method 200 further may comprise indicating to a user if the analyte sensor 102 presently is operated in the standard operation mode or in the economy operation mode.

The method 200 may comprises continuously detecting the analyte over a time span of at least one day, specifically over a time span of at least seven days. The method 200 may comprise continuously detecting the analyte by at least one of permanently evaluating sensor signals 130 of the analyte sensor 102 and repeatedly evaluating sensor signals 130 of the analyte sensor 102, specifically repeatedly evaluating sensor signals 130 acquired in regular or irregular time intervals.

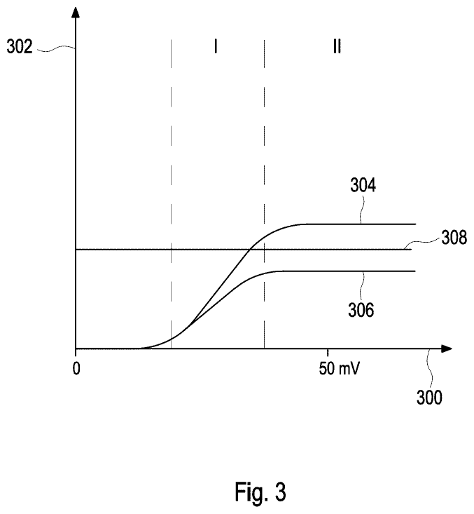

In FIG. 3, a current-voltage characteristic of an exemplarily embodiment of an analyte sensor 102 is depicted. On the horizontal axis 300, the potential between the working electrode 104 and the further electrode 106 is depicted. On the vertical axis 302 the current between the working electrode 104 and the further electrode 106 is depicted. Further, regime I, in which the analyte sensor 102 is operated in a kinetic-controlled region and a regime II, in which the analyte sensor 102 is operated in a diffusion-controlled region are depicted. Curve 306 represents the current-characteristic for a low glucose value, while curve 304 represents the current-characteristic for a high glucose value. When the exemplary analyte sensor 102 may be operated in the standard operation mode at a constant voltage of 50 mV, then the glucose level is proportional to the current. A variation of the potential within regime II may not influence the current. In the economy operation mode, the current may be fixed at a predetermined value as indicated by the line 308. The analyte sensor 102 may then be operated in the kinetic-controlled region and a variation of the potential may influence the current.

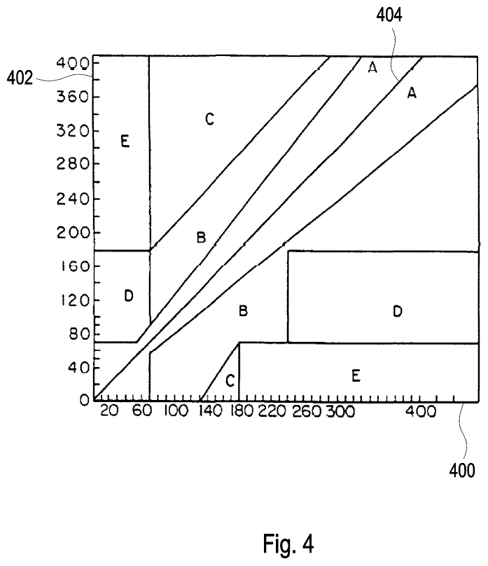

FIG. 4 shows an error grid analysis published in William L. Clarke et al., Evaluating Clinical Accuracy of Systems for Self-Monitoring of Blood Glucose, Diabetes Care, Vol. 10 No. 5, September-October 1987. A reference blood glucose level is shown on the x-axis 400. A corresponding value for the reference glucose level that is generated by a monitoring system is depicted on the y-axis 402. The diagonal 304 represents a perfect agreement between the reference blood glucose level and the value generated by a monitoring system. Data points lying in the area above and below the diagonal 404 represent overestimates and underestimates of the reference blood glucose level, respectively.

Based on several assumptions that are further discussed in the reference stated above, the grid is divided into five zones of varying degrees of accuracy and inaccuracy of monitored glucose estimations. Zone A represents glucose values that deviate from the reference by no more than 20% or are in the hypoglycemic range (<70 mg/dl) when the reference is also <70 mg/dl. Values falling within this range are clinically accurate in that they would lead to clinically correct treatment decisions. Upper and lower zone B represents values that deviate from the reference by >20% but would lead to appropriate or no treatment. Zone C values would result in overcorrecting acceptable glucose levels; such treatment might cause the actual blood glucose to fall below 70 mg/dl or rise above 180 mg/dl. Zone D represents “dangerous failure to detect and treat” errors. Actual glucose values are outside of the target range, but patient-generated values are within the target range. Zone E is an “erroneous treatment” zone. Patient-generated values within this zone are opposite to the reference values, and corresponding treatment decisions would therefore be opposite to that called for. In summary, values in zones A and B are clinically acceptable, whereas values in zones C, D, and E are potentially dangerous and therefore are clinically significant errors.

Particularly, zone A shows that with an increasing blood sugar level an increasing deviation between the value generated by a monitoring system, such as the analyte sensor system 100, and the reference blood glucose level is admissible, while still clinically acceptable decisions are made. That interpretation is further confirmed when taking into account the further zones, and particularly the zones C, D and E. The interpretation is specifically true for any reference blood glucose level above 200 mg/dl and further for a reference blood glucose level above 300 mg/dl. Any reference blood glucose level above these levels may be considered as being a hyperglycemic blood glucose level. As a result, the sensitivity of the analyte sensor 102 may be lower with an increasing reference blood glucose level and, still, clinically acceptable decisions will be made. This disclosure may make use of this effect by sacrificing the sensitivity sensor system 100 in the economy operation mode, specifically for a reference blood glucose level above 200 mg/dl and/or for a reference blood glucose level above 300 mg/dl.

An exemplary CGM curve of a patient is given in the document “Monitoring Technologies—Continuous Glucose Monitoring, Mobile Technology, Biomarkers of Glycemic Control” by Nihaal Reddy, BS, Neha Verma, MD, and Kathleen Dungan, MD, MPH in Feingold KR, Anawalt B, Boyce A, et al., Endotext [Internet] updated Aug 16, 2020. The patient shows a glucose level of typically less than 150 mg/dl. The patient further shows hyperglycemic episodes having a duration of about 4 hours. In these hyperglycemic episodes, the patient reaches blood glucose levels of above 400 mg/dl. Setting the blood glucose threshold value at a blood glucose level of 200 mg/dl may thus allow reducing the silver chloride consumption by 50% during these hyperglycemic episodes. Considering hyperglycemic episodes that last for 4 hours per day the silver chloride consumption may be reduced by more than 8%. The lower the blood glucose threshold value may be and the longer and/or higher the analyte excursions may be, the more silver chloride may be saved.

This may be confirmed by considering the formula

(Cᵢₛ−Cₜ)*S*tᵣₑₗ.

wherein Cᵢₛ is the glucose concentration in the hyperglycemic phases, Cₜ is the threshold value of the glucose concentration, S is the sensor sensitivity and tᵣₑₗ is the relative duration of the hyperglycemic phases.

While exemplary embodiments have been disclosed hereinabove, the present invention is not limited to the disclosed embodiments. Instead, this application is intended to cover any variations, uses, or adaptations of this disclosure using its general principles. Further, this application is intended to cover such departures from the present disclosure as come within known or customary practice in the art to which this invention pertains and which fall within the limits of the appended claims.

### LIST OF REFERENCE NUMBERS

100 analyte sensor system
102 analyte sensor
104 working electrode
106 further electrode
108 processor
110 memory
112 evaluation unit
114 layer
116 substrate
118 insulating material
120 window
122 electrode pad
124 contacting pad
126 membrane
128 electronic unit
130 sensor signal
132 standard sensor signal
134 economy sensor signal
200 method
202 monitoring step (step i.)
204 comparing step (step ii.)
206 switching step (step iii.)
208 further monitoring step (step iv.)
210 further comparing step (step v.)
212 further switching step (step vi.)
300 horizontal axis
302 vertical axis
304 curve
306 curve
308 line
400 horizontal axis
402 vertical axis
404 diagonal

What is claimed is:

1. A method of continuously in vivo detecting an analyte in a body fluid over a time span using an analyte sensor with a working electrode that performs an electrochemical detection reaction with the analyte and a further electrode having a redox material composition with silver and silver chloride, the method comprising the following steps:
i. monitoring a standard sensor signal derived by using the analyte sensor in a i. standard operation mode, wherein, in the standard operation mode, potentiostatic measurements are performed for detecting the analyte with the analyte sensor, wherein a potential of the working electrode with respect to the further electrode is set to a predetermined standard operating potential and wherein a current through the working electrode and the further electrode is determined; and
ii. comparing the standard sensor signal with a threshold, thereby determining if a change of the analyte sensor from the standard operation mode into an economy operation mode is required.

2. The method according to claim 1, wherein the analyte is selected from the group consisting of glucose, lactate and glutamate.

3. The method according to claim 1, further comprising:
iii. switching from the standard operation mode into the economy operation mode if, in step ii., it is determined that the change of the operation mode of the analyte sensor from the standard operation mode into the economy operation mode is required, wherein an economy sensor signal is generated in the economy operation mode.

4. The method according to claim 3, wherein the economy operation mode is performed either for a predetermined economy time span or until at least one condition for switching back from the economy operation mode into the standard operation mode is fulfilled.

5. The method according to claim 1, wherein, in the economy operation mode, at least one economy sensor signal is generated by performing galvanostatic measurements for detecting the analyte with the analyte sensor, wherein a current through the working electrode and the further electrode is set to a predetermined economy current value, wherein an operating potential of the working electrode with respect to the further electrode is determined.

6. The method according to claim 5, wherein the predetermined economy current value is chosen not to exceed an electric threshold current through the working electrode and the further electrode occurring when the standard sensor signal equals the threshold.

7. The method according to claim 1, further comprising:
iv. monitoring, when the analyte sensor is used in the economy operation mode, the economy sensor signal; and
v. comparing the economy sensor signal with an economy threshold, thereby determining if a change of the analyte sensor from the economy operation mode back into the standard operation mode is required.
vi. switching from the economy operation mode back into the standard operation mode if, in step v., it is determined that the change from the economy operation mode back into the standard operation mode is required.

8. The method according to claim 3, wherein the standard sensor signal and the economy sensor signal each are selected from the group consisting of electrical signals generated by the analyte sensor, electrical signals generated for regulating the analyte sensor, and secondary signals derived from electrical signals generated by the analyte sensor or for regulating the analyte sensor.

9. An analyte sensor system for in vivo continuously detecting an analyte in a body fluid over a measurement time span, comprising:
a. an electrochemical analyte sensor comprising a working electrode configured for performing an electrochemical detection reaction with the analyte, and a further electrode comprising a redox material composition comprising silver and silver chloride;
b. a processor and a memory storing instructions which when executed cause the processor to perform at least the following steps:
i. monitoring a standard sensor signal derived by using the analyte sensor in a standard operation mode, wherein, in the standard operation mode, potentiostatic measurements are performed for detecting the analyte with the analyte sensor, wherein a potential of the working electrode with respect to the further electrode is set to predetermined standard operating potential and wherein a current through the working electrode and the further electrode is determined; and
ii. comparing the standard sensor signal with a threshold, thereby determining if a change of the analyte sensor from the standard operation mode into an economy operation mode is required.

10. The analyte sensor system according to claim 9, wherein the further electrode comprises at least one layer comprising the redox material composition.

11. The analyte sensor system according to claim 9, wherein the further electrode is at least partially coated by an insulating material.

12. The analyte sensor system according to claim 11, wherein the insulating material leaves open at least one window through which the further electrode is accessible for the analyte.

13. The analyte sensor system according to claim 9, wherein the electrochemical analyte sensor comprises at least one membrane that is permeable for the analyte to be detected, the membrane at least partially surrounding the working electrode and the further electrode.

14. A computer program comprising instructions which, when the program is executed by the processor of an analyte sensor system, causes the analyte sensor system to perform the method according to claim 1.

15. A computer-readable storage medium comprising instructions which, when the instructions are executed by the processor of an analyte sensor system, cause the analyte sensor system to perform the method according claim 1.
